(19) United States
(12) Patent Application Publication
(10) Pub. No.: US 2024/0369533 A1
(43) Pub. Date: Nov. 7, 2024

(54) METHOD FOR DETERMINING EFFECT RELATIONSHIP BETWEEN VARIOUS SUBSTANCES AND CELLS, AND MICROWELL ARRAY CHIP

(71) Applicant: BGI SHENZHEN, Shenzhen (CN)

(72) Inventors: Yang LIU, Shenzhen (CN); Run XIE, Shenzhen (CN); Shiyu WANG, Shenzhen (CN); Kai GAO, Shenzhen (CN); Menghua LV, Shenzhen (CN); Ya LIU, Shenzhen (CN); Longqi LIU, Shenzhen (CN); Xun XU, Shenzhen (CN)

(21) Appl. No.: 18/774,933

(22) Filed: Jul. 17, 2024

Related U.S. Application Data
(63) Continuation of application No. PCT/CN2022/072517, filed on Jan. 18, 2022.

(51) Int. Cl. G01N 33/50; B01L 3/00; C12M 3/06

(52) U.S. Cl. CPC G01N 33/5023; B01L 3/502761; C12M 23/16; B01L 2200/0647; B01L 2200/16; B01L 2300/0636; B01L 2300/0829

(57) ABSTRACT

A method for determining an effect relationship between various substances and cells, including: providing a first droplet, a second droplet, a third droplet, and a microwell array chip, a plurality of microwell combinations being provided on the microwell array chip, fusing the first droplet and the second droplet on the microwell array chip, and performing cell culture; adding the third droplet into the microwell array chip, and fusing the third droplet with the first fused droplet subjected to the cell culture, to obtain a second fused droplet, performing demulsification, library construction, and sequencing, and determining an effect relationship between various substances and cells on the basis of the sequencing result.

### CROSS-REFERENCE TO RELATED APPLICATIONS

This application is a continuation of International Application No. PCT/CN2022/072517, filed on Jan. 18, 2022. The disclosure of the aforementioned application is hereby incorporated by reference in its entirety.

### TECHNICAL FIELD

The present disclosure relates to the field of medicine. In particular, the present disclosure provides a method for determining an effect relationship between various substances and cells, and a microwell array chip.

### BACKGROUND

Precision medicine refers to designing an optimal treatment plan for patients according to the difference between different people, hoping to achieve an optimal treatment effect and minimum side effects. With cancers are raging year by year, precise and individualized treatment has become one of the strategies to deal with the cancer heterogeneity. With the rapid development of sequencing technology over the last decade, scientists have discovered many genes related to carcinogenesis in different tissues, such as Myc, Ras, etc. By performing gene sequencing on a biopsy sample of a patient, information about possible oncogenic gene mutations can be obtained, so that treatment plans can be formulated accordingly. From a long-term perspective, personalized medicine provides more effective and targeted treatment through more accurate diagnosis, prevent the occurrence of certain disease, which saves treatment costs over current treatment measures. However, knowledge of the relationship between sequencing results and the response of cancer cells to a variety of external substances (including compounds, hormones, antibodies, etc. that can act on cells) is still very limited, so that the guidance of sequencing results for the formulation of personalized treatment regimens is also relatively limited at present.

In order to solve this problem, it is possible to conduct the in vitro response tests to additional substances (including compounds, hormones, antibodies, etc. that can act on cells) on the biopsy samples of patients to obtain the response effect of cancer cells to different substances, so as to assist in the formulation of personalized treatment plan. For the effectiveness of treatment, it is necessary to screen a large quantity of suspected effective substances, and the requirement of high throughput is more prominent in the screening of multiple drugs.

In recent years, DNA coding technology and sequencing technology are continuously developed, and DNA barcodes have been introduced into single cell sequencing to achieve high-throughput sequencing at a single cell level. At the same time, the realization of these high-throughput single cell sequencing is also inseparable from the development of microfluidic technology. Microfluidic technology is a technology capable of precisely controlling and manipulating microscale fluids, with the ability to miniaturize experiments performed in the laboratory to a chip with a scale of a few square centimeters, while it is also an interdisciplinary subject including physics, engineering, chemistry and biology, etc. Since the micro-structure of microfluidic chips is comparable to the size of single cells, microfluidic chips are considered to be the most promising high-throughput single cell analysis platform in the field of biology, especially in single cell-related research. Analysis at the single cell level has important research significance for early diagnosis and treatment of major diseases and drug screening, and has become a hot spot in recent years. Currently reported methods for drug screening at the single cell level cannot achieve high-throughput drug screening. Therefore, a method that can study the effects of different drugs on single cells at the transcriptome level is needed to achieve large-scale drug screening in vitro, which is helpful for screening multiple drugs with synergistic effects, and provides assistance for personalized precision medicine and drug combination.

### SUMMARY

The present disclosure aims to solve the technical problems existing in the prior art at least to some extent. To this end, the present disclosure proposes a method for determining an effect relationship between various substances and cells, and a microwell array chip. By using this method, the effect relationship of a plurality of substances can be studied at a single cell level, which helps to achieve the high-throughput screening of combined substances, and is of great significance for the study of the combined substances at the transcriptome level and the guidance of drug combination.

In one aspect of the present disclosure, the present disclosure provides a method for determining an effect relationship between various substances and cells. According to an embodiment of the present disclosure, the method includes:
step 1: providing a first droplet, a second droplet, a third droplet, and a microwell array chip, wherein:
   the first droplet is a mixed droplet containing a plurality of different molecule-encoding droplets, each of the plurality of molecule-encoding droplets containing one substance, and an encoding nucleic acid molecule and an index magnetic bead that matching the one substance;
   the second droplet is a droplet containing a single cell;
   the third droplet is a droplet containing a single sequencing magnetic bead, a cell lysis solution, and a fragmentation reagent; and
   the microwell array chip has a plurality of microwell combinations disposed thereon, each of the plurality of microwell combinations comprising one large microwell and a plurality of small microwells adjacent to and in communication with the large microwell, and the large microwell having a greater diameter than each of the plurality of small microwells;
step 2: adding the second droplet into the microwell array chip and allowing the second droplet to fall into the large microwell, and adding the first droplet to the microwell array chip and allowing the first droplet to fall into the plurality of small microwells;
step 3: fusing the first droplet and the second droplet to obtain a first fused droplet, wherein the first fused droplet occupies the large microwell and the plurality of small microwells are vacated, and performing cell culture on the microwell array chip;
step 4: adding, subsequent to the completion of the cell culture, the third droplet into the plurality of vacated small microwells, fusing the third droplet with the first fused droplet subjected to the cell culture to obtain a second fused droplet, and collecting the second fused droplet, wherein cell rupture occurs under an action of a cell lysis solution in the third droplet, and wherein nucleic acid molecules in the cells and the encoding nucleic acid molecule are captured by the sequencing magnetic bead, and an index sequence on the index magnetic bead is cleaved under an action of a fragmentation reagent in the third droplet, the index sequence on the index magnetic bead being also captured by the sequencing magnetic bead; and
step 5: demulsifying the second fused droplet, collecting the sequencing magnetic bead, performing library construction and sequencing on the nucleic acid and the index sequence carried on the sequencing magnetic bead, and determining the effect relationship between various substances and cells based on a sequencing result.

In the method according to an embodiment of the present disclosure, different substances are encoded and labeled with encoding nucleic acid molecules (also referred to as “molecular codes”) to facilitate subsequent analysis of sequencing results. Both substance droplets and magnetic bead droplets are small droplets, and cell droplets are large droplets. The microwell array chip has communicated microwells with different diameters, the microwells with small diameters can capture small droplets, and the microwells with large diameters can capture large droplets.

Firstly, the second droplet with a larger diameter (also referred to as a “cell droplet”) is added to the chip and falls into the large microwell. Then, the first droplet with a small diameter (also referred to as a “substance droplet”) is added to the chip, each substance droplet will randomly fall into one small microwell, then one cell droplet is fused with a plurality of substance droplets to complete the addition process, and the chip is taken out after being incubated in an incubator for a certain period of time. Due to the interfacial tension, the fused large droplet will occupy the large microwell and leaving the position of the small microwells vacant for subsequent loading of sequencing magnetic bead droplets. After substance treatment is completed, the third droplet (a small droplet wrapped with the sequencing magnetic bead, the cell lysis solution, and the fragmentation reagent, also referred to as a “magnetic bead droplet”) is added to the small microwells within the chip and fused with the large droplet to complete cell lysis and capture of mRNA, the molecular codes, and the index sequence. The fused droplets are collected, the magnetic beads are recovered after demulsification, and the nucleic acid information and index sequence information carried on the magnetic beads are subjected to a subsequent single cell library construction process.

Since a plurality of substance droplets act together on one cell droplet, and a plurality of sequencing magnetic beads (the quantity is consistent with a quantity of substance droplets) capture nucleic acid information after cell culture, in order to know whether different sequencing magnetic beads act on the same cell droplet, an index magnetic bead carrying the index sequence is added to each substance droplet. The sequencing magnetic beads can capture the index sequences, and based on the type of the index sequences, it can be known whether they are derived from the same cell droplet, and thus a plurality of substances acting on the same cell can be known, which is helpful to achieve high-throughput screening of combined substances, and is of great significance for the study of combined substances at the transcriptome level and the guidance of drug combination.

According to an embodiment of the present disclosure, the method for determining an effect relationship between various substances and cells may further have the following additional technical features.

According to an embodiment of the present disclosure, each of the molecule-encoding droplets includes 3 to 8 index magnetic beads, the index sequences on the respective index magnetic beads being different.

According to an embodiment of the present disclosure, the fragmentation reagent is suitable for fragmenting disulfide bonds.

According to an embodiment of the present disclosure, the fragmentation reagent is selected from dithiothreitol, tri(2-carbonylethyl)phosphine hydrochloride, tri(3-hydroxypropyl)phosphine, and/or β-mercaptoethanol.

According to an embodiment of the present disclosure, the second droplet and the third droplet are obtained by sorting via a sorting chip.

According to an embodiment of the present disclosure, said fusing is electrofusion or chemical fusion.

According to an embodiment of the present disclosure, the large microwell has a diameter of 80 μm to 100 μm and a depth of 60 μm to 80 μm; and the small microwells have a diameter of 40 μm to 60 μm and a depth of 60 μm to 80 μm.

According to an embodiment of the present disclosure, the sequencing magnetic bead is suitable for capturing the nucleic acid molecules and the index sequence.

According to an embodiment of the present disclosure, prior to step 2, the microwell array chip provided in step 1 is subjected to a surface plasma treatment for bonding a groove for flow of the droplets in the microwell array chip to the large microwell and the plurality of small microwells to capture the droplets.

According to an embodiment of the present disclosure, said collecting the second fused droplet includes: flipping the microwell array chip by 180° to allow openings of the large microwell and the plurality of small microwells to face upward, and adding oil to the microwell array chip, enabling the second fused droplet to flow out of the large microwell into a collection container.

In yet another aspect of the present disclosure, the present disclosure provides a microwell array chip. According to an embodiment of the present disclosure, the microwell array chip includes: a microwell array layer, wherein a plurality of microwell combinations are provided on the microwell array layer, each of the plurality of microwell combinations includes one large microwell and a plurality of small microwells adjacent to and in communication with the large microwell, and the large microwell having a greater diameter than each of the plurality of small microwells; and a channel layer laminated on the microwell array layer, wherein a groove is provided on the channel layer, openings of the large microwell and the plurality of small microwells facing the groove. As previously described, by using the microwell array chip according to an embodiment of the present disclosure. the effect relationship of combined substances can be studied at a single cell level, which helps to achieve the high-throughput screening of combined substances, and is of great significance for the study of the combined substances at the transcriptome level.

According to an embodiment of the present disclosure, the large microwell has a diameter of 80 μm to 100 μm and a depth of 60 μm to 80 μm; and the plurality of small microwells have a diameter of 40 μm to 60 μm and a depth of 60 μm to 80 μm.

According to an embodiment of the present disclosure, the groove is connected to the large microwell or the small microwells through a chemical bond.

According to an embodiment of the present disclosure, the microwell array chip is configured to perform the method for screening the combined substances described above.

The method designed in the present disclosure uses molecular codes to encode substances under different conditions, and to wrap a single cell, a nucleic acid capturing magnetic bead and a substance and a corresponding code into one same droplet in combination with the microfluidic technology, which can achieve high-throughput screening of combined substances at the single cell level through single cell sequencing technology, thereby improving the screening efficiency of combined substance and reducing the consumption of manpower and material resources.

Additional aspects and advantages of the present disclosure will be set forth in part in the description which follows and, in part, will be apparent from the description, or may be learned by practice of the present disclosure.

### BRIEF DESCRIPTION OF DRAWINGS

The foregoing and/or additional aspects and advantages of the present disclosure will become apparent and readily understood from the following description of the embodiments, taken in conjunction with the accompanying drawings, in which:

FIG. 1 shows a schematic flowchart of a method for determining an effect relationship between various substances and cells according to an embodiment of the present disclosure;

FIG. 2 shows a top view of a structure of a microwell array chip according to an embodiment of the present disclosure;

FIG. 3 shows a schematic diagram of a channel layer structure according to an embodiment of the present disclosure;

FIG. 4 shows a side view of a microwell array chip structure according to an embodiment of the present disclosure;

FIG. 5 shows a schematic flowchart of droplet preparation according to an embodiment of the present disclosure;

FIG. 6 shows a schematic flowchart of droplet capturing and fusion according to an embodiment of the present disclosure;

FIG. 7 shows a physical picture (a) of a droplet capturing a large droplet and a physical picture (b) of a droplet capturing a small droplet according to an embodiment of the present disclosure, the scale is 100 μm;

FIG. 8 shows a schematic diagram of the sequencing magnetic bead capturing sequence information and a constitution of a substance code according to an embodiment of the present disclosure;

FIG. 9 shows a fragment profile after specific amplification of an index sequence according to an embodiment of the present disclosure, with a distinct characteristic peak at around 170 bp; and

FIG. 10 shows a fragment profile after specific amplification of a substance coding sequence according to an embodiment of the present disclosure, with a distinct characteristic peak at around 130 bp.

### DESCRIPTION OF EMBODIMENTS

Embodiments of the present disclosure will be described in detail below. The embodiments described below are exemplary and are intended to explain the present disclosure and are not to be construed as limiting the present disclosure.

It should be noted that the terms “first”, “second” are only used for descriptive purposes, and can not be construed as indicating or implying relative importance or implicitly indicating the quantity of technical features indicated. Thus, features defined with “first” or “second” may explicitly or implicitly include one or more of the feature. Furthermore, in the description of the present disclosure, the meaning of “plurality” is two or more, unless indicated otherwise.

The present disclosure provides a method for determining an effect relationship between various substances and cells, and a microwell array chip, each of which will be described in detail below.

### The Method for Determining an Effect Relationship Between Various Substances and Cells

In one aspect of the present disclosure, the present disclosure provides a method for determining an effect relationship between various substances and cells. The specific type of the term “substance” in the present disclosure is not strictly restricted, and may be a compound, hormone, antibody, etc. that can or cannot produce an effect on the body. For the convenience of understanding and description, in the present disclosure, a “substance” may be embodied generally as a “drug”.

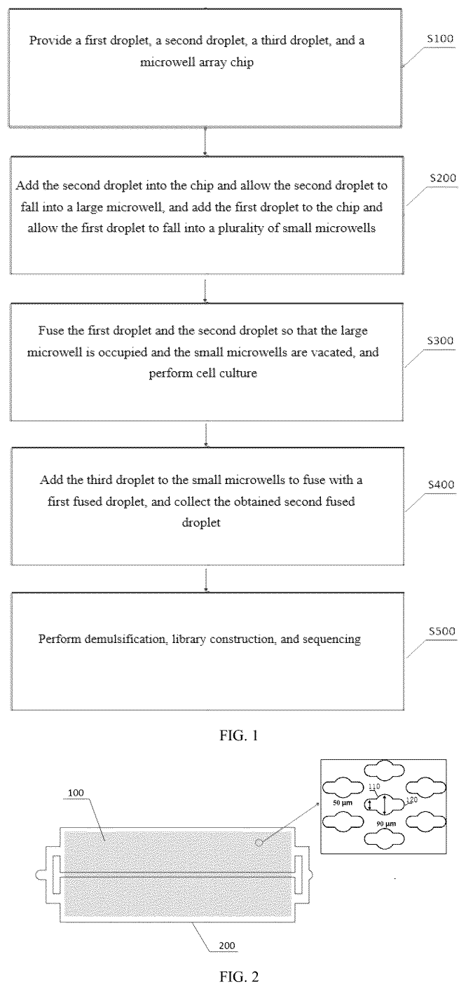

According to an embodiment of the present disclosure, with reference to FIG. 1, the method for determining an effect relationship between various substances and cells includes that:

S100 a first droplet, a second droplet, a third droplet, and a microwell array chip are provided.

In this embodiment, a first droplet, a second droplet, a third droplet and a microwell array chip are provided, wherein the first droplet is a mixed droplet containing a plurality of different molecule-encoding droplets, each of the the plurality of molecule-encoding droplets containing one substance and an encoding nucleic acid molecule (also referred to as “molecular code”) that matching the one substance; the second droplet is a droplet containing a single cell, the third droplet is a droplet containing a single sequencing magnetic bead (for capturing the nucleic acid), a cell lysis solution and a fragmentation reagent, the microwell array chip has a plurality of microwell combinations disposed thereon, each of the plurality of microwell combinations including one large microwell and a plurality of small microwells adjacent to and in communication with the large microwell.

Fluorescent encoding technology refers to the use of the color of fluorescent dye to encode solutions of different drugs and generate droplets, which are then mixed with the droplet wrapped with a single cell to achieve single cell-based drug screening. However, this method is limited by fluorescent species and detection devices, which makes it difficult to screen a large quantity of drugs and lacks information on gene expression inside the cell.

In the present disclosure, using molecular code design, based on exogenous sequence information introduced in international patent WO2021147069A1, there are M×N types of codes (M=4¹⁰, N=4¹⁰) in total. By synthesizing the sequence as a drug code, a drug under one condition corresponds to one code. For example, the molecular code contains a fixed sequence UMI, a specific sequence and a magnetic bead capturing sequence.

According to an embodiment of the present disclosure, a method of preparing the first droplet includes the following: a certain amount of molecular codes are mixed with corresponding substances and index magnetic beads, and then injected into a droplet generation chip to generate molecule-encoding droplets with a uniform size, and so on to generate droplets with different molecular codes. Finally, the generated droplets are collected and mixed in one same collection tube to complete the preparation of substance droplets. The molecular codes are added for the purpose of distinguishing different substances. The reason for introducing index magnetic beads is mainly because a plurality of sequencing magnetic beads will be captured when the sequencing magnetic bead droplets are finally captured, so that the final fused droplet contains a plurality of sequencing magnetic beads. If the index sequence is not added, it will make it impossible to determine which two sequencing magnetic beads are from the same droplet in the final sequencing, and the mRNA information captured by the sequencing magnetic beads cannot be grouped together, that is, it will be impossible to determined which drugs act on the same cell. This makes single cell sequencing data incomplete and unusable. In order to solve this problem, an index magnetic bead carrying an index sequence is introduced, and the index magnetic beads can be cleaved to generate the index sequences under the action of the fragmentation reagent. Since each index magnetic bead is different, this makes each final large droplet contain a variety of different index sequences. By sequencing analysis, it can be determined that the index sequences captured on which sequencing magnetic beads are of the same type, thus, it can be considered that these sequencing magnetic beads are derived from the same droplet, and then the mRNA information and molecular code information captured by the these sequencing magnetic beads are combined into one by an algorithm, so that a complete single cell sequencing can be achieved. In particular, each of the molecule-encoding droplets includes 3 to 8 index magnetic beads, the index sequences on the respective index magnetic beads being different.

The method of preparing the second droplet includes the following: a certain amount of cell suspension is injected into the droplet generation chip, the cell concentration is adjusted to make the ratio of single cell wrapped in the droplet higher, and ensure the double wrapping rate at a lower level, and then the generated droplets are sorted by a sorting chip using dielectrophoresis and other methods to obtain the droplet containing a single cell.

A method of preparing the third droplet includes the following: a certain amount of sequencing magnetic bead suspension (obtained by resuspending the sequencing magnetic beads using the cell lysis solution and the fragmentation reagent) is injected into the droplet generation chip, and the concentration of the sequencing magnetic beads is adjusted to make the ratio of the single sequencing magnetic bead wrapped in the droplet higher, and ensure the double wrapping rate at a relatively low level, and then the generated droplets are sorted by a sorting chip using dielectrophoresis and other methods to obtain the droplet containing a single sequencing magnetic bead.

It should be noted that references to “large” and “small” in the “large microwell” and “small microwells” described herein refer to a diameter of the microwells, a diameter of the large microwell being larger than a diameter of the small microwells. According to an embodiment of the present disclosure, the large microwell has a diameter of 80 μm to 100 μm and a depth of 60 μm to 80 μm; and the small microwells have a diameter of 40 μm to 60 μm and a depth of 60 μm to 80 μm. Thus, the large microwell can capture single cell droplets, the small microwells can capture substance droplets and magnetic bead droplets.

According to an embodiment of the present disclosure, the fragmentation reagent is suitable for fragmenting disulfide bonds. The index sequence is linked to the magnetic bead through a disulfide bond, and the disulfide bond is cleaved by using the fragmentation reagent, so that the index sequence carried on the index magnetic bead is cleaved from the magnetic bead. Since the sequencing magnetic bead contains a sequence matching the index sequence, the cleaved index sequence can be captured, so as to facilitate subsequent sequencing and classification, and learn multiple substances acting on the same cell. According to a specific embodiment of the present disclosure, the fragmentation reagent is selected from dithiothreitol (DTT), tri(2-carbonylethyl)phosphine hydrochloride (TCEP), tri (3-hydroxypropyl)phosphine (THPP) and/or β-mercaptoethanol. Thus, disulfide bonds can be broken specifically without affecting the nucleic acid molecular structure.

S200 Firstly, the second droplet is added into the chip and falls into a large microwell, and then the first droplet is added to the chip and falls into a plurality of small microwells.

In this embodiment, firstly, the second droplet is added into the microwell array chip and falls into the large microwell, and then the first droplet is added to the microwell array chip and falls into the plurality of small microwells.

According to an embodiment of the present disclosure, prior to step S200, the microwell array chip provided in step S100 is subjected to a surface plasma treatment for bonding a groove for flow of the droplets in the microwell array chip to the large microwell and the small microwells to capture the droplets.

As used herein, the term “bonding” refers to a technique in which two pieces of homogeneous or heterogeneous semiconductor material with clean and atomically flat surfaces are directly bonded under certain conditions after surface cleaning and activation, and the wafers are bonded together by van der Waals forces, molecular forces or even atomic forces.

The surface of the cured chip (also referred to as “PDMS substrate”) has a certain adhesion force, and a pair of formed PDMS substrates can be bonded naturally without any treatment by means of intermolecular attraction, but the bonding strength is limited, and it is prone to leakage. The surface of plasma-treated PDMS has —OH groups with hydrophilic property which replace —CH groups, so that the surface of PDMS exhibits very strong hydrophilic property. The treated two layers of PDMS are attached, and the following reaction occurs between the Si—OH of the two surfaces: 2Si—OH®Si—O-Si+2H₂O. A strong Si—O bonding is formed between the two PDMS layers, thereby completing an irreversible bonding between the two.

S300 The first droplet and the second droplet are fused and occupy the large microwell and the small microwells are vacated for cell culture.

In this embodiment, the first droplet and the second droplet are fused to obtain a first fused droplet, wherein the first fused droplet occupies the large microwell and the small microwells are vacated, and cell culture is performed on the microwell array chip.

Due to the interfacial tension, the fused first fused droplet will mainly occupy the large microwell, leaving the position of the small microwells vacant for the subsequent loading of sequencing magnetic bead droplets.

It should be noted that the present disclosure does not strictly limit the fusion manner of the two droplets. For example, it can be realized by using an electric field to destroy the stability of the interface, or by using a chemical reagent such as perfluorobutanol, and the specific manner can be flexibly selected according to practical requirements.

S400 The third droplet is added to the small microwells to fuse with the first fused droplet, and the obtained second fused droplet is collected.

In this embodiment, the third droplet is added, subsequent to the completion of the cell culture, into the vacated small microwells, and the third droplet will be fused with the first fused droplet subjected to the cell culture, wherein cell rupture occurs under an action of the cell lysis solution in the third droplet, and the nucleic acid molecules in the cells and the encoding nucleic acid molecule are captured by the sequencing magnetic bead, and at the same time, an index sequence on the index magnetic bead is cleaved under an action of the fragmentation reagent in the third droplet, and is also captured by the sequencing magnetic bead to obtain a second fused droplet, and the second fused droplet is collected.

After the drug treatment is completed, a small droplet wrapped with the sequencing magnetic bead, the cell lysis solution and the fragmentation reagent is added into the chip, and fused with the first fused droplet to obtain the second fused droplet, thereby completing the capture of cell lysis, mRNA, molecular code and index sequences.

S500 Demulsification, library construction, and sequencing are performed.

In this embodiment, the second fused droplet is demulsified, the sequencing magnetic beads are collected, library construction and sequencing are performed on the nucleic acid and index sequence carried on the sequencing magnetic beads, and the effect relationship between various substances and cells is determined based on the sequencing results.

According to an embodiment of the present disclosure, said collecting the second fused droplet includes: flipping the microwell array chip by 180° to allow openings of the large microwell and the plurality of small microwells to face upward, and adding oil into the microwell array chip, enabling the second fused droplet to flow out of the large microwell into a collection container.

The microwell array chip includes a microwell array layer and a channel layer which are arranged in a laminated manner, wherein the large microwell and the small microwells are arranged on the microwell array layer, and openings of the microwells face a groove for liquid flow which is provided on the channel layer.

Since the density of an aqueous phase is lower than that of an oil phase, the second fused droplet floats above the groove and cannot be taken away by adding oil into the groove. Thus, the chip needs to be flipped by 180° to allow the openings of the large microwell and the small microwells face upward so that the second fused droplet will float in the groove, and the droplet detached from the microwell can be pushed out with oil and collected in a centrifuge tube. Then the magnetic beads are recovered after demulsification for a subsequent single cell library construction process, and a corresponding relationship between a single cell and a drug is obtained through sequencing information resolution, thereby achieving high-throughput screening of drugs at the single cell level.

The method for screening drugs of the present disclosure may also have the following advantages:
1) High-throughput: by additionally introducing the molecular codes, the encoding of multiple drug conditions (including drug species, drug concentration, etc.) can be realized; and by additional introducing the index magnetic beads, a plurality of substances acting on the same cell can be distinguished, so as to facilitate the study of the effect of multiple substances on cells, which is helpful for the drug combination;
2) Time-and-effort saving: by using the droplet microfluidic technology, it can not only reduce the amount of reagents, but also avoid cumbersome manual operations and improve the screening efficiency;
3) High accuracy: since the present disclosure is directed to single cell analysis, the corresponding relationship between single cell transcriptome and drugs can be obtained by introducing molecular code in combination with a single cell sequencing means, so that the effects of drugs on single cell transcriptome under different conditions can be displayed more accurately, which is of great research value.

### Microwell Array Chip

In yet another aspect of the present disclosure, the present disclosure provides a microwell array chip. According to an embodiment of the present disclosure, referring to FIG. 2, the microwell array chip includes: the microwell array layer 100 and the channel layer 200.

A plurality of microwell combinations are provided on the microwell array layer 100, each of the plurality of microwell combinations comprises one large microwell 110 and a plurality of small microwells 120 adjacent to and in communication with the large microwell 110, and the large microwell 110 having a greater diameter than each of the plurality of small microwells 120. Both drug droplets and magnetic bead droplets are small droplets, and cell droplets are large droplets. The microwell array layer has communicated microwells with different diameters, microwells with small diameters can capture small droplets, and microwells with large diameters can capture large droplets.

According to an embodiment of the present disclosure, the large microwell has a diameter of 80 μm to 100 μm and a depth of 60 μm to 80 μm; and the small microwells have a diameter of 40 μm to 60 μm and a depth of 60 μm to 80 μm. Thus, the large microwell can capture single cell droplets, the small microwells can capture drug droplets and magnetic bead droplets.

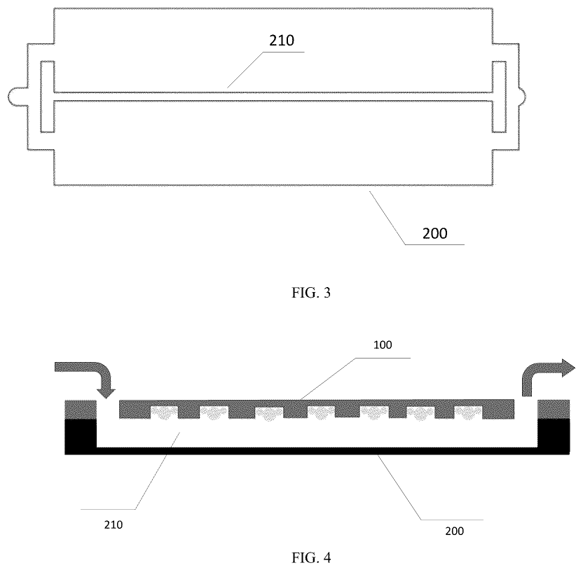

Referring to FIGS. 3 and 4, the channel layer 200 is laminated on the microwell array layer 100, and the groove 210 is provided on the channel layer, with the openings of the large microwell 110 and the plurality of small microwells 120 facing the groove 210. Since the microwells of the microwell array layer and the groove have been subjected to surface plasma treatment in advance, the two can be bonded together so as to avoid the leakage of the added liquid, droplets are added into the groove, and since the droplets are lighter than oil, the microwells can capture the droplets under the effect of buoyancy.

According to an embodiment of the present disclosure, the groove 210 is connected to the large microwell 110 or the plurality of small microwells 120 through a chemical bond. Thus, droplets that flow into the groove can be captured by the microwells.

Specifically, before adding the droplets, two holes are punched on the microwell array layer respectively, namely, a liquid inlet and a liquid outlet, both of which are opposite to the groove. The droplets are added into the groove through the liquid inlet, and the liquid will be captured by the microwells, and then oil is added into the groove through the liquid inlet, so as to wash away the non-captured droplets in the groove, and the droplets are sucked out from the liquid outlet.

According to an embodiment of the present disclosure, the microwell array chip is configured to perform the method for screening the drugs described above. The features and advantages described above with respect to the method of screening drugs are equally applicable to the microwell array chip and will not be described further herein.

The implementation of the present disclosure will be explained with reference to the following examples. Those skilled in the art will understand that the following examples are merely for illustrating the present disclosure and should not to be construed as limiting the scope of the present disclosure. Where specific techniques or conditions are not specified in the examples, they are performed according to techniques or conditions described in the literature in the art or according to the product specification. The reagents or instruments used, without indicating the manufacturer, are conventional products commercially available.

### Example 1

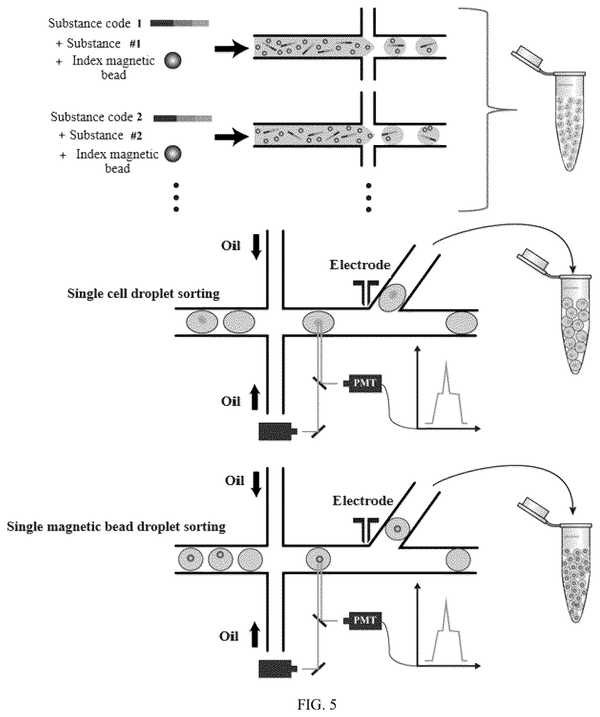

Step 1. A droplet generation device similar to CN209144161U was used in this example, in which the droplet generation chip was replaced with the chip involved in patent WO2020063864 A1, and the syringe was replaced with a BD 30 ml syringe. The schematic diagram of droplet generation was shown in FIG. 5, and cell droplets, drug droplets and sequencing magnetic bead droplets were generated with reference to the following steps.

### 1. Design of Molecular Code

The molecular code used in the present disclosure was based on the exogenous sequence information introduced in the international patent WO2021147069A1 invention patent, and has M×N types of codes (M=410, N=410). By synthesizing the sequence as a drug code, a drug under one condition corresponds to one code.

### (2) Preparation of the Drug Droplets

A certain amount of molecular codes, corresponding drugs and index magnetic beads carrying the index sequences were mixed and then injected into a droplet generation chip to generate molecule-encoding droplets with a uniform size, and so on to generate droplets with different molecular codes. Finally, the generated droplets were collected and mixed in one same collection tube to complete the preparation of drug droplets.

### (3) Preparation of the Single Cell Droplet

A certain amount of cell suspension was injected into the droplet generation chip, the cell concentration was adjusted to make the ratio of single cell wrapped in the droplet higher and ensure the double wrapping rate at a lower level, and then the generated droplets were sorted by a cell sorting chip using dielectrophoresis and other methods to obtain the droplet containing a single cell.

### (4) Preparation of the Sequencing Magnetic Bead Droplets

A certain amount of sequencing magnetic bead suspension (obtained by resuspending the sequencing magnetic beads using the cell lysis solution) was injected into the droplet generation chip, and the concentration of the sequencing magnetic beads was adjusted to make the ratio of the single sequencing magnetic bead wrapped in the droplet relatively high, and ensure the double wrapping rate at a relatively low level, and then the generated droplets were sorted by a sorting chip using dielectrophoresis and other methods to obtain the droplet containing a single sequencing magnetic bead.

Step 2, A microwell chip as shown in FIGS. 1 to 4 was used to capture droplets in this example. The microwell chip included a microwell array layer and a channel layer, the microwell array layer had 28,800 interconnected microwells (with a diameter of 90 μm and 50 μm respectively), and a depth of the microwells was 70 μm, thus ensuring that only one large droplet can be captured by one large microwell and only one small droplet can be captured by one small microwell, and achieving 1:1 pairing of droplets. Since the density of the aqueous phase is lower than that of the oil phase, in this case the droplets float on an upper layer of the oil phase. In order to capture the droplets, the chip was needed to be flipped by 180° in practical use, making the openings of the microwells face down, and the microwell array layer is bonded with the channel layer shown in FIG. 2 through surface plasma treatment, and in this case the channel contains more than 25,000 effective microwells. A hole punch with a diameter of 1.5 millimeter was used to punch two holes on the microwell array layer, namely a liquid inlet and a liquid outlet.

The specific operating steps were as follows:

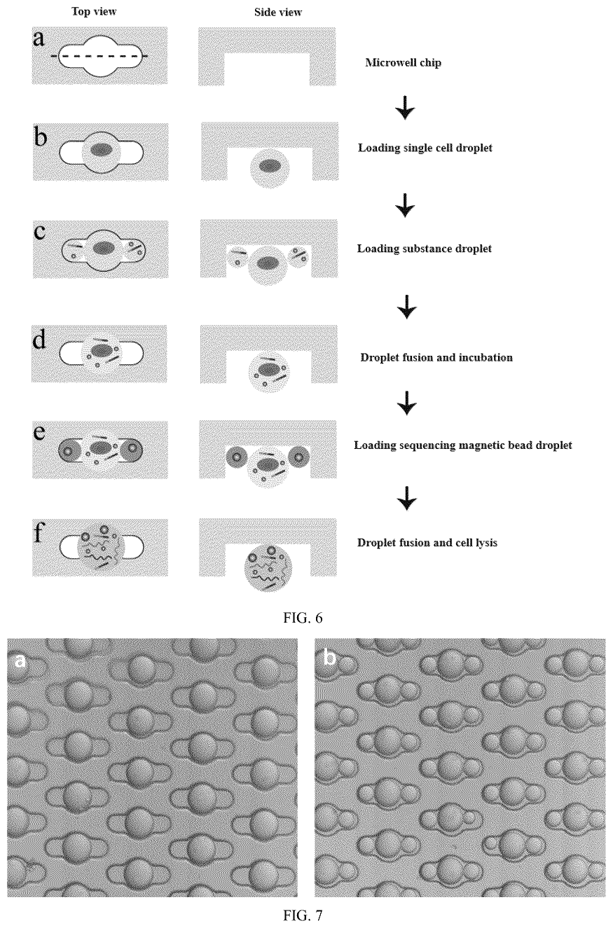

A large droplets (with a diameter of 90 μm) wrapping single cells were first generated and the cell droplets were loaded into the microwell array chip (a of FIG. 6), allowing the large microwell to capture the cell droplets, as shown in b of FIG. 6 and a of FIG. 7.

Then, small droplets (with a diameter of 50 μm) that generate a plurality of types of mixed wrapped substances, molecular codes and index magnetic beads were loaded into the microwell array chip, and different types of small droplets were randomly captured by the small microwells, so that each group of microwells randomly captures two substance small droplets and one single cell large droplet, and after the microwells capture the droplets, oil was injected to push away the redundant droplets, as shown in c of FIG. 6 and b of FIG. 7, and the droplet capturing efficiency was above 95%.

The chip was placed on a shaker and shaked slightly to make the same group of droplets collide, and at the same time, the droplets were fused with an corona machine or a chemical reagent perfluorobutanol, as shown in d of FIG. 6. At this time, the fused droplets randomly contain one or two substances and corresponding molecular codes, and index magnetic beads (the index sequence was not cleaved, because no fragmentation reagent was added), and the fusion makes the positions of two small droplets vacant, and the fusion efficiency was above 80%.

After incubating the droplets for a period of time, droplets containing one sequencing magnetic bead were added, so that each group of microwells will capture two sequencing magnetic bead droplets, and the excess droplets were pushed out with oil. The droplet status at this time was shown in e of FIG. 6.

Finally, after the droplets were fused, since the sequencing magnetic bead droplets contain the cell lysis solution and the index sequence fragmentation reagent, the cell will be lysed, and at the same time, the index sequence on the index magnetic bead will be cleaved by the fragmentation reagent. In this way, the sequencing magnetic bead can capture the mRNA generated by cell lysis, the index sequence cleaved from the index magnetic beads and the molecular code corresponding to the substance.

Step 3, after the droplet incubation was completed, the microfluidic chip was taken out, and then the sequencing magnetic bead droplet was injected, at this time, the droplet will be captured again by the vacated small microwell, and then excess droplets were pushed out with oil, and at this time, the state of the droplet was as shown in e of FIG. 6. Finally, the process of droplet fusion in step 2 was repeated, and the state of the droplets at this time was shown in f of FIG. 6.

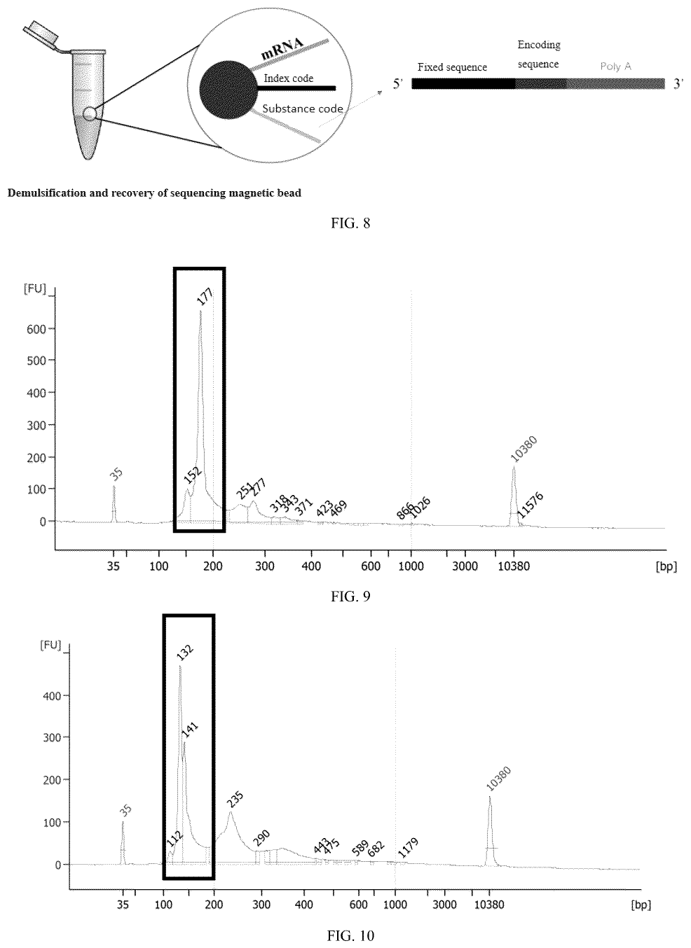

Step 4, in order to recover the secondary fused droplets, the microwell array chip needed to be flipped by 180°, then the droplets detached from the microwells were pushed out of the chip with oil, and the droplets were collected in a centrifuge tube. As shown in FIG. 8, since the sequencing magnetic bead droplets contain cell lysis solution, once the droplets are fused for the second time, the cell will be lysed to release intracellular mRNA, and at this time, the sequencing magnetic beads will capture all the nucleic acid information including the mRNA, the molecular code and the index sequence. Finally, the BGI single cell sequencing process was performed, and the magnetic beads labeled with the same type of index sequence are classified as originating from the same droplet by bioinformatics analysis. Among them, a reaction solution for respectively purifying the secondary specific amplification index sequence and the secondary specific amplification substance code was verified by gel running, and the experimental results thereof are shown in FIGS. 9 and 10, with distinct peaks at about 170 bp and 130 bp, respectively, demonstrating that the present disclosure can detect the molecular code of the combined substance via single cell sequencing technology, and the introduction of the index sequence was to label a plurality of sequencing magnetic beads in the same droplet.

In the description of this specification, references to descriptions of the terms “an embodiment”, “some embodiments”, “examples”, “specific examples”, or “some examples”, etc. mean that a specific feature, structure, material, or characteristic described in connection with the embodiment or example is included in at least one embodiment or example of the present disclosure. In this specification, schematic representations of the above-mentioned terms are not necessarily directed to the same embodiment or example. Furthermore, the specific features, structures, materials, or characteristics described may be combined in any suitable manner in any one or more embodiments or examples. Furthermore, those skilled in the art can integrate and combine different embodiments or examples and features of the different embodiments or examples described in this specification without conflicting with each other.

Although the embodiments of the present disclosure have been shown and described above, it will be understood that the above embodiments are exemplary and cannot be construed as limiting the present disclosure, and those skilled in the art can make changes, modifications, substitutions and variations to the above embodiments within the scope of the present disclosure.

What is claimed is:

1. A method for determining an effect relationship between various substances and cells, comprising:
step 1: providing a first droplet, a second droplet, a third droplet, and a microwell array chip, wherein:
the first droplet is a mixed droplet containing a plurality of different molecule-encoding droplets, each of the plurality of molecule-encoding droplets containing one substance, and an encoding nucleic acid molecule and an index magnetic bead that matching the one substance;
the second droplet is a droplet containing a single cell;
the third droplet is a droplet containing a single sequencing magnetic bead, a cell lysis solution, and a fragmentation reagent; and
the microwell array chip has a plurality of microwell combinations disposed thereon, each of the plurality of microwell combinations comprising one large microwell and a plurality of small microwells adjacent to and in communication with the large microwell, and the large microwell having a greater diameter than each of the plurality of small microwells;
step 2: adding the second droplet into the microwell array chip and allowing the second droplet to fall into the large microwell, and adding the first droplet to the microwell array chip and allowing the first droplet to fall into the plurality of small microwells;
step 3: fusing the first droplet and the second droplet to obtain a first fused droplet, wherein the first fused droplet occupies the large microwell and the plurality of small microwells are vacated, and performing cell culture on the microwell array chip;
step 4: adding, subsequent to the completion of the cell culture, the third droplet into the plurality of vacated small microwells, fusing the third droplet with the first fused droplet subjected to the cell culture to obtain a second fused droplet, and collecting the second fused droplet, wherein cell rupture occurs under an action of a cell lysis solution in the third droplet, and wherein nucleic acid molecules in the cells and the encoding nucleic acid molecule are captured by the sequencing magnetic bead, and an index sequence on the index magnetic bead is cleaved under an action of a fragmentation reagent in the third droplet, the index sequence on the index magnetic bead being also captured by the sequencing magnetic bead; and
step 5: demulsifying the second fused droplet, collecting the sequencing magnetic bead, performing library construction and sequencing on the nucleic acid and the index sequence carried on the sequencing magnetic bead, and determining the effect relationship between various substances and cells based on a sequencing result.

2. The method according to claim 1, wherein each of the plurality of molecule-encoding droplets comprises 3 to 8 index magnetic beads, the index sequences on respective index magnetic beads being different.

3. The method according to claim 1, wherein the fragmentation reagent is suitable for fragmenting disulfide bonds.

4. The method according to claim 3, wherein the fragmentation reagent is selected from dithiothreitol, tri(2-carbonylethyl)phosphine hydrochloride, tri(3-hydroxypropyl)phosphine, and/or β-mercaptoethanol.

5. The method according to claim 1, wherein the second droplet and the third droplet are obtained by sorting via a sorting chip.

6. The method according to claim 1, wherein said fusing is electrofusion or chemical fusion.

7. The method according to claim 1, wherein:
the large microwell has a diameter of 80 μm to 100 μm and a depth of 60 μm to 80 μm; and
the plurality of small microwells have each a diameter of 40 μm to 60 μm and a depth of 60 μm to 80 μm.

8. The method according to claim 1, wherein the sequencing magnetic bead is suitable for capturing the nucleic acid molecules and the index sequence.

9. The method according to claim 1, wherein, prior to step 2, the microwell array chip provided in step 1 is subjected to a surface plasma treatment for bonding a groove for flow of the droplets in the microwell array chip to the large microwell and the plurality of small microwells to capture the droplets.

10. The method according to claim 1, wherein said collecting the second fused droplet comprises:
flipping the microwell array chip by 180° to allow openings of the large microwell and the plurality of small microwells to face upward, and adding oil into the microwell array chip, enabling the second fused droplet to flow out of the large microwell into a collection container.

11. A microwell array chip, comprising:
a microwell array layer, wherein a plurality of microwell combinations are provided on the microwell array layer, each of the plurality of microwell combinations comprising one large microwell and a plurality of small microwells adjacent to and in communication with the large microwell, and the large microwell having a greater diameter than each of the plurality of small microwells; and
a channel layer laminated on the microwell array layer, wherein a groove is provided on the channel layer, openings of the large microwell and the plurality of small microwells facing the groove.

12. The microwell array chip according to claim 11, wherein the large microwell has a diameter of 80 μm to 100 μm and a depth of 60 μm to 80 μm.

13. The microwell array chip according to claim 11, wherein the plurality of small microwells have each a diameter of 40 μm to 60 μm and a depth of 60 μm to 80 μm.

14. The microwell array chip according to claim 11, wherein the groove is connected to the large microwell or the plurality of small microwells through a chemical bond.

15. The microwell array chip according to claim 11, wherein the microwell array chip is configured to perform a method for determining an effect relationship between various substances and cells, comprising:
step 1: providing a first droplet, a second droplet, a third droplet, and a microwell array chip, wherein:
the first droplet is a mixed droplet containing a plurality of different molecule-encoding droplets, each of the plurality of molecule-encoding droplets containing one substance, and an encoding nucleic acid molecule and an index magnetic bead that matching the one substance;
the second droplet is a droplet containing a single cell;
the third droplet is a droplet containing a single sequencing magnetic bead, a cell lysis solution, and a fragmentation reagent; and
the microwell array chip has a plurality of microwell combinations disposed thereon, each of the plurality of microwell combinations comprising one large microwell and a plurality of small microwells adjacent to and in communication with the large microwell, and the large microwell having a greater diameter than each of the plurality of small microwells;
step 2: adding the second droplet into the microwell array chip and allowing the second droplet to fall into the large microwell, and adding the first droplet to the microwell array chip and allowing the first droplet to fall into the plurality of small microwells;
step 3: fusing the first droplet and the second droplet to obtain a first fused droplet, wherein the first fused droplet occupies the large microwell and the plurality of small microwells are vacated, and performing cell culture on the microwell array chip;
step 4: adding, subsequent to the completion of the cell culture, the third droplet into the plurality of vacated small microwells, fusing the third droplet with the first fused droplet subjected to the cell culture to obtain a second fused droplet, and collecting the second fused droplet, wherein cell rupture occurs under an action of a cell lysis solution in the third droplet, and wherein nucleic acid molecules in the cells and the encoding nucleic acid molecule are captured by the sequencing magnetic bead, and an index sequence on the index magnetic bead is cleaved under an action of a fragmentation reagent in the third droplet, the index sequence on the index magnetic bead being also captured by the sequencing magnetic bead; and
step 5: demulsifying the second fused droplet, collecting the sequencing magnetic bead, performing library construction and sequencing on the nucleic acid and the index sequence carried on the sequencing magnetic bead, and determining the effect relationship between various substances and cells based on a sequencing result.
